(19) United States
(12) Patent Application Publication
(10) Pub. No.: US 2007/0215507 A1
(43) Pub. Date: Sep. 20, 2007

(54) Modular surgical tray and delivery system

(76) Inventors: William S. Glenn, Cordova, TN (US); Robin L. Turner, Memphis, TN (US); John M. B. Ford, Cordova, TN (US); Christopher A. Bagozzi, Memphis, TN (US); Tatika Jones, Memphis, TN (US); Randall J. Troutman, Cordova, TN (US); Sherry D. Hernandez, Biloxi, MS (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/378,292

(22) Filed: Mar. 20, 2006

(51) Int. Cl. A61B 19/02; B65D 1/34

(52) U.S. Cl. 206/438; 206/557

(57) ABSTRACT

A modular surgical tray and delivery system is disclosed. According to one embodiment of the present invention, the surgical tray system comprises a surgical tray having a base portion and a plurality of lower endcaps secured to the base portion. The system also includes a surgical tray cover having a top portion and a plurality of upper endcaps secured to the top portion. According another embodiment, a method of manufacture of a surgical tray system is disclosed. The method includes the steps of: (1) providing a surgical tray having a base portion; (2) securing at least one lower endcap to the base portion of the surgical tray; (3) providing a surgical tray cover having a top portion; and (4) securing at least one upper endcap to the top portion of the surgical tray cover.

### BACKGROUND OF THE INVENTION

1. Field of the Invention

The present invention generally relates to a surgical tray, and, more particularly, to a modular surgical tray and delivery system.

2. Description of the Related Art

Trays that can be used for the storage and transportation of surgical instruments are known. For example, U.S. Pat. No. 4,643,303 to Arp et al., the disclosure of which is incorporated by reference, discloses a modular sterilizing system that facilitates the sterilization, storage, protection, handling and presentment of surgical instruments and devices. This patent discloses the use of a basket and several modules, each of which being designed for a specific surgical instrument. U.S. Pat. No. 5,424,048, the disclosure of which is incorporated by reference, also discloses a modular sterilization tray system.

### SUMMARY OF THE INVENTION

It is a technical advantage of the present invention that a surgical tray system is disclosed. It is another technical advantage of the present invention that a method of manufacture of a surgical tray system is disclosed. It is still another technical advantage of the present invention that endcaps may be provided on the base portion of the surgical tray, and on the top portion of the surgical tray cover.

A surgical tray system is disclosed. According to one embodiment of the present invention, the surgical tray system comprises a surgical tray having a base portion and a plurality of lower endcaps secured to the base portion. The system also includes a surgical tray cover having a top portion and a plurality of upper endcaps secured to the top portion.

The at least one lower endcap may be removeably secured to the base portion, and the at least one upper endcap may be removeably secured to the top portion. The lower and upper endcaps may comprise a polyphenylsulfone high temperature resin. The lower and upper endcaps may be at least partially covered with an overmolding, and the overmolding may comprise a thermoplastic elastomer. The surgical tray cover may comprises a window. The surgical tray system may comprise an identification device.

A method of manufacture of a surgical tray system is disclosed. According to one embodiment, the method includes the steps of: (1) providing a surgical tray having a base portion; (2) securing at least one lower endcap to the base portion of the surgical tray; (3) providing a surgical tray cover having a top portion; and (4) securing at least one upper endcap to the top portion of the surgical tray cover.

The at least one lower endcap may be removeably secured to the base portion, and the at least one upper endcap may be removeably secured to the top portion. The lower and upper endcaps may comprise a polyphenylsulfone high temperature resin. The lower and upper endcaps may be at least partially covered with an overmolding, and the overmolding may comprise a thermoplastic elastomer. The surgical tray cover may comprises a window. The surgical tray system may comprise an identification device.

A surgical tray system according to another embodiment of the invention is disclosed. The surgical tray system comprises a surgical tray that comprises a base portion and at least one lower endcap removeably secured to the base portion. The system further comprises a surgical tray cover that comprises a top portion and at least one upper endcap removeably secured to the top portion. The upper and lower endcaps are at least partially covered with an overmolding. The cover may include at least one latch, and the latch may be a slide latch.

The lower and upper endcaps may comprise a polyphenylsulfone high temperature resin The overmolding may comprise a thermoplastic elastomer. The surgical tray cover may further comprise a window. The surgical tray system may further comprise an identification device.

### BRIEF DESCRIPTION OF THE DRAWINGS

For a more complete understanding of the present invention, the objects and advantages thereof, reference is now made to the following descriptions taken in connection with the accompanying drawings in which:

FIG. 1 is an illustration of a surgical tray according to one embodiment of the present invention; and

FIG. 2 is an illustration of a cover for a surgical tray of FIG. 1 according to one embodiment of the present invention.

### DETAILED DESCRIPTION OF PREFERRED EMBODIMENTS

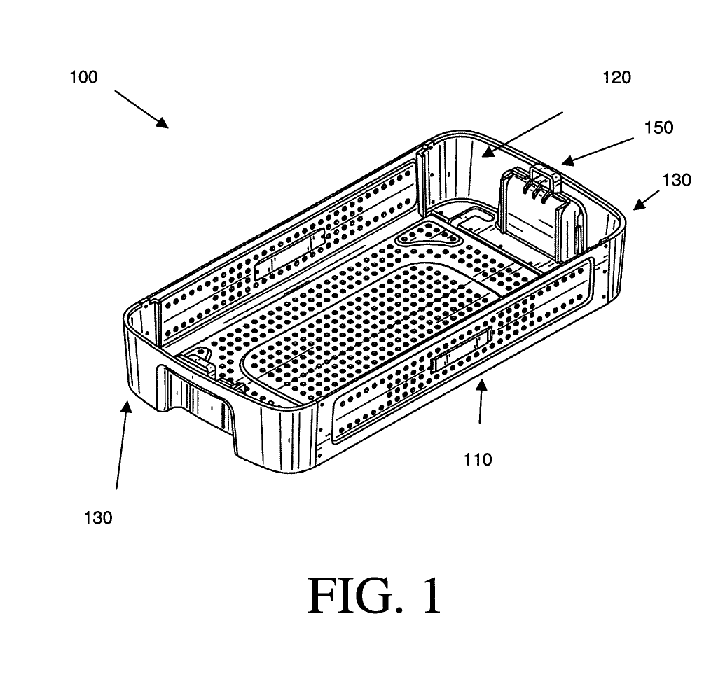

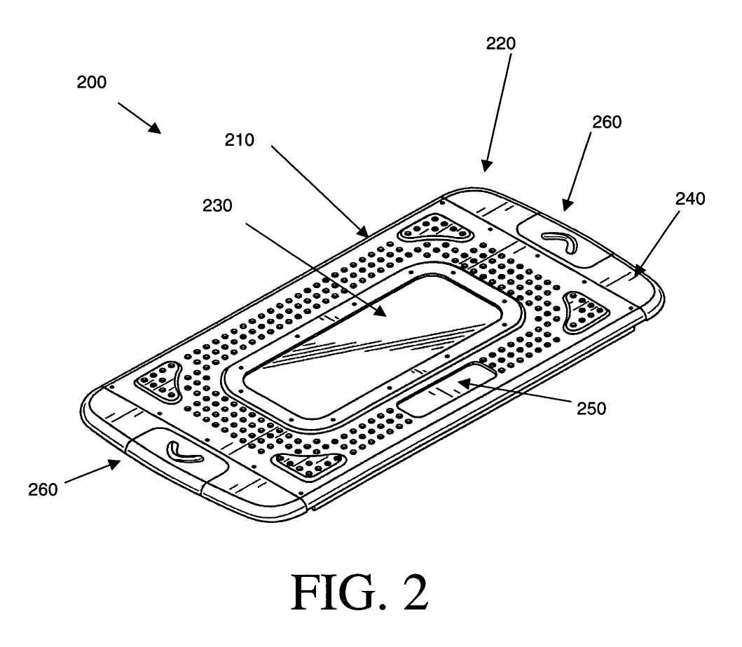

Preferred embodiments of the present invention and their advantages may be understood by referring to FIGS. 1-2, wherein like reference numerals refer to like elements.

Referring to FIG. 1, surgical tray 100 according to one embodiment of the present invention is provided. Surgical tray 100 includes base portion 110 and lower endcaps 120. In one embodiment, base portion 110 may be made of a metal, such as aluminum, stainless steel, etc. Lower endcaps 120 may be made of Radel® polyphenylsulfone high temperature resin, available from Solvay Advanced Polymers, L.L.C., Alpharetta, Ga.

Lower endcaps 120 may be secured to base portion 110 in a suitable fashion. In one embodiment, lower endcaps 120 are riveted to base portion 110. Other suitable mechanisms for securing lower endcaps 120 to base portion 110 may be used.

Lower endcaps 120 may be removed and replaced as necessary.

Lower endcaps 120 may be formed by injection molding in order to allow for complex case design. In one embodiment, lower endcaps 120 provide a molded handle.

Lower endcaps 120 may be at least partially covered with overmolding 130, such as such as a thermoplastic elastomer. Overmolding 130 may be provided to give a cushioned feel and a better grip. Overmolding 130 also provides a handing point for surgical tray 100 following sterilization.

In addition, overmolding 130 may act as a bumper to prevent damage. As a notable amount of damage to existing surgical trays is located at the ends of the surgical trays, the overmolding 130 will absorb much of the abuse seen throughout the distribution environment.

In one embodiment, overmolding 130 may be mechanically secured or adhered to its lower endcaps 120 to prevent overmolding 130 from being “peeled” off of lower endcaps 120. Although adhesives may be used, it is preferable to use mechanical fastening.

An example of a suitable overmolding is Santoprene® thermoplastic elastomer, available from Advanced Elastomer Systems, L.P.; 388 South Main Street; Akron, Ohio 44311.

Lower endcaps 120 may be provided with latch receiving portion 150. Latch receiving portion 150 is provided to engage the latch engaging portion (not shown). Details of the latching mechanism will be discussed in greater detail below.

Referring to FIG. 2, matching cover 200, according to one embodiment of the present invention, for surgical tray 100 is provided. Cover 200 includes top portion 210, upper end caps 220, and window 230. In one embodiment, similar to base portion, 110, top portion 210 may be made of a metal, such as aluminum.

Upper endcaps 220 may be shaped to interact with lower endcaps 120. Similar to lower endcaps 120, upper endcaps may be made of Radel® polyphenylsulfone high temperature resin and may be at least partially covered with overmolding 240. Upper endcaps 220 may be secured to upper portion 210 in a suitable fashion. In one embodiment, upper endcaps 220 are riveted to upper portion 210. Other suitable mechanisms for securing upper endcaps 220 to upper portion 210 may be used. As with lower endcaps 120, upper endcaps may be removed and replaced as necessary.

Window 230 may be provided in cover 200 to allow for product visibility and product presentation. In one embodiment, window 230 allows for sterility indicators that are placed by the window to be viewed.

Window 230 may be made of a suitable transparent material. In one embodiment, transparent Radel® R polyphenylsulfone, available from Solvay Advanced Polymers, L.L.C., Alpharetta, Ga. may be used.

Surgical case 100 or cover 200 may be provided with identification device 250. Identification device 250 is used to quickly identify the contents of surgical tray 100. Identification device 250 may be any suitable identification mechanism, such as a color code, a graphic, a barcode, or a Radio Frequency Identification (“RFID”) tag.

In one embodiment, cover 200 maybe provided with a label (not shown) containing product information. The label may be replaced as the contents change.

In one embodiment, latch mechanism 260 is provided to secure cover 200 to tray 100. Latch 260 may be provided in at least one of upper endcaps 220. Latch 260 may also be located at the edge of cover 200, preferably in the center of cover 200.

Latch 260 may be a biased towards closure. Accordingly, in one embodiment, latch 260 may be spring-loaded so that it is in the closed position by default, and will return to the closed position if released.

Latch 260 may be implemented in a variety of ways. In one embodiment, latch 260 may be implemented as a slide latch. In this embodiment, latch 260 may be opened by sliding latch 260 outwardly. This causes the engaging portion (not shown) of latch 260 to disengage from latch engaging portion 150 of surgical tray 100, illustrated in FIG. 1. Cover 200 may then be removed from surgical tray 100.

Latch 260 may be engaged by placing cover 200 over surgical tray 100 and applying pressure on cover 200. A taper (not shown) in the engaging portion (not shown) causes latch 260 to move slightly outward so that engaging portion can engage surgical tray 100. Once in position, latch 260 will return inwardly because it is so biased.

Only one latch 260 may be provided for cover 200. This may allow latch 260 to be opened and cover 200 to be removed from surgical tray 100 by using only one hand.

Both cover 200 and tray 100 may include embossed areas in order to facilitate stackability. For example, a bottom surface of tray 100 may be stacked on a top surface of cover 200.

Although lower endcaps 120 and upper endcaps 220 are shown as being secured to the width of surgical tray 100 and cover 200, respectively, lower endcaps 120 and upper endcaps 220 may be provided on the lengths of surgical tray 100 and cover 200, respectively, In addition, a single endcap along the periphery of surgical tray 100 or cover 200 may be provided.

Other embodiments, uses, and advantages of the present invention will be apparent to those skilled in the art from consideration of the specification and practice of the invention disclosed herein. The specification and examples should be considered exemplary only.

What is claimed is:

1. A surgical tray system, comprising:
a surgical tray having a base portion and at least one lower endcap secured to the base portion; and
a surgical tray cover having a top portion and at least one upper endcap secured to the top portion.

2. The surgical tray system of claim 1, wherein the at least one lower endcap is removeably secured to the base portion, and the at least one upper endcap is removeably secured to the top portion.

3. The surgical tray system of claim 1, wherein the surgical tray cover comprises at least one latch, and the base portion comprises a latch receiving portion.

4. The surgical tray system of claim 3, wherein the latch is a slide latch.

5. The surgical tray system of claim 1, wherein the lower and upper endcaps are at least partially covered with an overmolding.

6. The surgical tray system of claim 4, wherein the overmolding comprises a thermoplastic elastomer.

7. The surgical tray system of claim 1, wherein the surgical tray cover further comprises a window.

8. The surgical tray system of claim 1, further comprising an identification device.

9. A method of manufacture of a surgical tray system, comprising:
providing a surgical tray having a base portion;
securing at least one lower endcap to the base portion of the surgical tray;
providing a surgical tray cover having a top portion; and
securing at least one upper endcap to the top portion of the surgical tray cover.

10. The method of claim 9, wherein the at least one lower endcap is removeably secured to the base portion, and the at least one upper endcap is removeably secured to the top portion.

11. The method of claim 9, wherein the lower and upper endcaps comprise a polyphenylsulfone high temperature resin

12. The method of claim 9, further comprising:
at least partially covering the lower and upper endcaps with an overmolding.

13. The method of claim 12, wherein the overmolding comprises a thermoplastic elastomer.

14. The method of claim 9, further comprising:
providing a window in the surgical tray cover.

15. The method of claim 9, further comprising:
providing an identification device.

16. The method of claim 9, further comprising:
providing a latch in the surgical tray cover.

17. The method of claim 16, wherein the latch is a sliding latch.

18. A surgical tray system, comprising:
a surgical tray comprising:
a base portion; and
at least one lower endcap removeably secured to the base portion; and
a surgical tray cover comprising:
a top portion; and
at least one upper endcap removeably secured to the top portion;
wherein the lower and upper endcaps are at least partially covered with an overmolding.

19. The surgical tray system of claim 18, wherein the lower and upper endcaps comprise a polyphenylsulfone high temperature resin

20. The surgical tray system of claim 18, wherein the overmolding comprises a thermoplastic elastomer.

21. The surgical tray system of claim 18, wherein the surgical tray cover further comprises a window.

22. The surgical tray system of claim 18, further comprising an identification device.

23. The surgical tray system of claim 18, wherein the surgical tray cover further comprises a latch.

24. The surgical tray system of claim 23, wherein the latch is a slide latch.
